(19) United States
(12) Patent Application Publication
(10) Pub. No.: US 2023/0384290 A1
(43) Pub. Date: Nov. 30, 2023

(54) Cellular Response Analysis Method

(71) Applicant: BIOMEMS ANALYTICS LLC, South Bend, IN (US)

(72) Inventors: Michael J. Pugia, Pagosa Springs, CO (US); Jason M. Kulick, South Bend, IN (US)

(21) Appl. No.: 18/249,087

(22) PCT Filed: Oct. 15, 2021
(86) PCT No.: PCT/US21/55253

§ 371 Date: Apr. 14, 2023

Related U.S. Application Data
(60) Provisional application No. 63/092,951, filed on Oct. 16, 2020.

(51) Int. Cl. G01N 33/50; B01L 3/00

(52) U.S. Cl. CPC G01N 33/5005; B01L 3/5085; B01L 2300/0645; B01L 2300/0681

(57) ABSTRACT

A method and system for cellular response analysis whereby cells are captured into an analyte detection microwell with a size exclusion filter using an affinity agent to capture an analyte, such as a specific cell or biomolecule, by a cleavable linkage arm connected to the size exclusion filter where the affinity captured analyte, such as a cell or biomolecule, does not pass through the size exclusion filter at the bottom of the microwell, but analyte, not affinity captured, do pass along with debris from the sample.

### CROSS-REFERENCE TO RELATED APPLICATION

This application is the United States national phase of International Application No. PCT/US21/55253 filed Oct. 15, 2021, and claims priority to U.S. Provisional Patent Application No. 63/092,951, filed Oct. 16, 2020, the disclosures of which are hereby incorporated by reference in their entireties.

### BACKGROUND OF THE INVENTION

### Field of the Invention

The present disclosure relates to a method of analysis of heterogenic cell populations arrayed into microwells where the presence or absence of response to stimuli is measured for one or more cellular phenotype which can be selectively released using electrochemical current to selectively remove cells and biomarkers from any given well.

### Description of Related Art

Cellular analysis is important in many medical applications for diagnosis and treatment of many diseases. The detection of biomolecules that are bound, inside or secreted by living cells, is highly desirable for determining cellular function, cell type, response to treatments, cellular differentiation, cell growth, cell death, and many other important responses. These responses can predict whether a cell is trying to adapt and survive or if a disease is becoming resistant, atypic, or malignant. This kind of environmental or chronic stress is often not possible to test in the host until the tissues or cells are isolated or removed.

Current methods for analysis of cellular responses must be done in-vivo using cell cultures or in-vitro using animal models. As a result, the phenotype and genotype of a single cell are not often measured. Recent discoveries are showing that a single cell can cause significant biological effects, such as a cancer stem cell ability to promote metastatic disease or an induced pluripotent stem cell ability to restore cell function.

However, current methods for analysis of cellular response require many cells, typically greater than 10\hat{ }6 cells per analysis (Pugia Anal Chem 2021). Additionally, these cells are often in altered cultured states not reflecting the true in-vivo or in-vitro conditions in an organism or biome. Primary un-altered cells often do not grow rapidly, and it requires lengthy times to produce enough new cells to measure biomolecules produced, bound, or consumed. This presents an issue in the selection of a pure cell clone by the current methods. These methods do not truly represent each primary cell but rather are a heterogenic population of many similar variants as it is necessary to grow many cells to obtain enough biological materials for measurement. Clearly, a new method for analysis of cellular responses from fewer cells, ideally a single cell, is needed.

Another problem with current analysis of cellular response is that the cells express many different types of biomolecules and low expression levels must be detected and separated from un-important expression levels. Finding the correct pattern of biomolecules' expression currently requires continued growing of a large cell population to generate enough materials for testing and having methods capable of measuring such expression. Additionally, variant selection occurs in this practice and the most common biomolecules expressed by all variants are detected before rare expressions. Therefore, there is a need for methods to measure all types of biomolecules at single cell levels to characterize each unique clone across its cellular response.

The analysis of cellular responses after single cell capture by size exclusion filtration and microscopic imaging is capable of measuring presence of one or more characteristic biomolecules of a cellular phenotype. However, the ability to multiplex and quantitate the presence of more than a few characteristic phenotypes is complex and time consuming.

A new cellular response methodology was recently demonstrated (Pugia et al. Anal Chem 2019) whereby multiple biomolecules could be quantitated using filtration membranes to isolate small numbers of cells when combined with detection methods such as mass spectrometric immunoassays (MS-IA), electrochemical immunoassays (EC-IA), optical immunoassays (OP-IA), and polymerase chain reaction (PCR) methods. These methods were compatible and allowed the same cellular sample to be analyzed multiple times with different methods providing the additional information needed for cellular phenotyping. The MS-IA, OP-IA, EC-IA methods were non-destructive to DNA and RNA of the cells and allowed both cellular phenotyping and genotyping from the same sample (Pugia Anal Chem 2021). However, these methods still required many cells, typically greater than 10\hat{ }4 prokaryotic cells or then 20 eukaryotic cells per analysis.

In IBRI's PCT/US2020/055931 (the “IBRI PCT”), which is incorporated by reference in its entirety, an analyte detection microwell is described for electrochemical detection of target analytes that replaces the filtration membrane. The analyte detection microwell includes a size exclusion filter, electrochemical detector, and affinity agents for a target analyte capture and detection, and operates under low hydrodynamic force without clogging with debris. The affinity agent for detection is attached to a reagent capable of generating an electrochemical label. The affinity agent for capture is attached to a reagent capable of binding a surface in the microwell. The electrochemical label is detected by a working electrode and a reference electrode placed in the microwell to measure a label formed by the affinity agent for detection.

The IBRI PCT design allows precise containment of the small sample volumes into an analyte detection microwell without the loss of detection liquid, exposure to the environment, or the need for extraction and delivery into an analyzer. This allows a concentration of signal needed for single cell sensitivity for electrochemical immunoassays (EC-IA), while still compatible with single cell microscopic imaging by optical immunoassays (OP-IA), and mass spectrometric immunoassays (MS-IA), and polymerase chain reaction (PCR) methods.

However, in practice, the issues of this approach become apparent as the isolation of single cells from one specific microwell for MS and PCR analysis was not possible. While compatible surfactants can be found to lyse both prokaryotes and eukaryotes for polymerase chain reaction (PCR), they interfere with downstream mass spectrometric immunoassay (MS-IA) methods. These methods lysed all cells in all wells and the content of individual wells cannot be isolated from all other wells. Isolation of cells or biomolecules from one specific microwell for separate analysis by multiple methods was not possible.

A solution is needed for a means to improve the selective release of biomolecules from an individual well for further analysis without surfactants or damage to cells and biomarkers.

### SUMMARY OF THE INVENTION

An object of the present disclosure is a method for analysis of cells arrayed into microwells where the presence or absence of response to stimuli is measured for one or more cellular phenotypes and the content of a specific microwell can be released by breaking of a linkage arm to selectively remove cells from any given well. The invention relates to methods and kits for analysis of cells. The invention also relates to analysis methods to measure biomolecules produced, bound, or consumed by cells, which, in term, can measure the response of cells and/or biological active compounds.

Another object of the present disclosure is to allow a convenient and accurate way to free cells or a biomolecule in an analyte detection microwell by breaking a linker arm with a disulfide S—S bond, which is attached to a capture affinity agent in an analyte detection microwell that can release the cell or biomarker being broken upon application of current into the microwell with an electrode.

Another object of the present disclosure is to allow a convenient and accurate way to identify an analyte detection microwell needing removal of cell or biomolecule content by electrochemical detection to allow selection of a microwell for application of a current with an electrode which does not free the captured cells or biomolecule by breaking the disulfide S—S bond.

A non-limiting embodiment of the present disclosure allows collection of cells or biomolecule contents of an analyte detection microwell by delivering the freed contents into a sample collection chamber by application of a hydrodynamic force. Hydrodynamic forces are used in driving the freed cells or biomolecule contents in a microwell through one or more pores of the analyte detection microwell into a sample collection area.

A non-limiting embodiment of the present disclosure is a method disclosed herein to include: 1) to capture a cell or biomolecule into an analyte detection microwell; 2) detection of the cell or biomolecule in the analyte detection microwell; 3) selection of an analyte detection microwell for application of a current to free a captured cell or biomolecule; and 4) collection of the contents of the analyte detection microwell into a sample collection area via a hydrodynamic force. The analyte detection microwell includes a size exclusion filter, electrochemical detector, and affinity agents for target analyte capture and detection as described in IBRI PCT.

Further preferred and non-limiting embodiments or examples are set forth in the following numbered clauses.

Clause 1: A method of determining cellular response comprising: introducing a complex sample into a microwell, the microwell having a size exclusion filter, the complex having an analyte; introducing a stimuli into the microwell; measuring a response from the analyte; analyzing the response; and releasing the analyte from the microwell.

Clause 2: The method of clause 1, further comprising applying a voltage in the microwell, wherein the voltage allows the analyte to exit the microwell via the size exclusion filter, and wherein the analyte comprises cells or biomolecules.

Clause 3: The method of any of clauses 1-2, wherein the response from the analyte triggers applying the voltage in the microwell.

Clause 4: The method of any of clauses 1-3, wherein the analyte is captured by an affinity agent, wherein the affinity agent is connected to a cleavable linkage arm, the cleavable linkage arm connected to the microwell.

Clause 5: The method of any of clauses 1-4, wherein a voltage separates the cleavable linkage arm and the capture agent.

Clause 6: The method of any of clauses 1-5, wherein the analyte is selectively captured in the microwells by an affinity agent prior to exiting the microwell.

Clause 7: The method of any of clauses 1-6, wherein the response from the analyte is measured before adding the stimuli into the microwell.

Clause 8: The method of any of clauses 1-7, further comprising measuring molecules and/or biomolecules from the analyte in the presence of one or more compounds to determine therapeutic response or the biological activity of the analyte.

Clause 9: The method of any of clauses 1-8, further comprising determining conditions of the analyte that increases or decreases biomolecule production depending on the response of the analyte.

Clause 10: The method of any of clauses 1-9, further comprising diagnosing the analyte depending on the response of the analyte.

Clause 11: The method of any of clauses 1-10, wherein the response comprises a signal from an antibody, biomolecule, or a cell.

Clause 12: The method of any of clauses 1-11, where in a response is measured in the presence of one or more compounds, and wherein the response determines a therapeutic response or the biological activity of the analyte.

Clause 13: The method of any of clauses 1-12, wherein the released analyte is intact.

These and other features and characteristics of the present disclosure, as well as the methods of operation and functions of the related elements of structures and the combination of parts will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 shows a schematic view of an analyte detection microwell according to a non-limiting embodiment of the invention.

FIGS. 2A-2C show schematic views of an analyte detection microwell according to a non-limiting embodiment of the invention.

FIG. 3 shows a schematic view of a system using a vacuum pump with a micro-filtration sensor with microwells according to a non-limiting embodiment of the invention.

FIG. 4 shows a schematic view of a holder from FIG. 3 according to a non-limiting embodiment of the invention.

### DESCRIPTION OF THE INVENTION

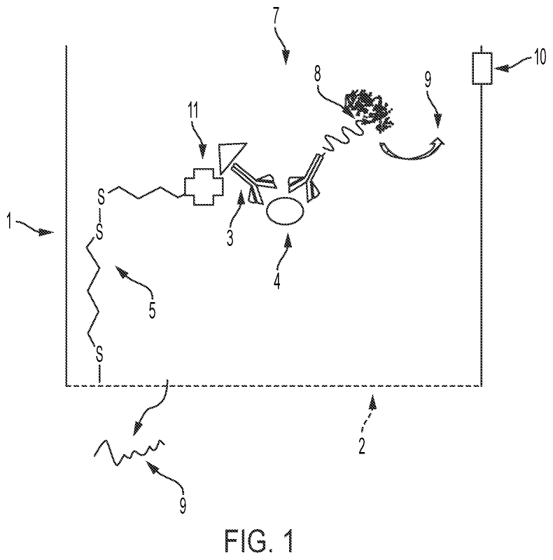

FIG. 1 illustrates a non-limiting embodiment of the present disclosure, whereby cells are captured into an analyte detection microwell (1) with a size exclusion filter (2) using an affinity agent (3) to capture an analyte (4), such as a specific cell or biomolecule, by a cleavable linkage arm (5) connected to the size exclusion filter (2). In a non-limiting embodiment, the affinity captured analyte (4), such as a cell or biomolecule, does not pass through the size exclusion filter (2) at the bottom of the microwell (1) but the analyte (4), not affinity captured, does pass along with debris (6) from the sample. A signal generating reagent (8) is added to the microwell (1) and the response of the analyte (4) is measured by a signal generated (9) using an electrode (10). The response is used to trigger application of a current into the microwell (1) with the electrode (10) to the break linkage arm (5) and allow release of the analyte (4) through the size exclusion filter (2).

In non-limiting embodiments or examples, one or more additional assay results are produced after releasing the analyte (4) and are measured by additional signal generating reagents (8). In other embodiments, the assay result produced prior to releasing of the analyte (4) is measured by a signal generating reagent (8) capable of producing an electrochemical signal. In one non-limiting example, the signal generating reagent (8) is an antibody capable of generating a signal as a measure of cell response. In non-limiting examples, the signal generating reagent (8) is a direct binding molecule or reagents capable of generating an optical, mass spectrometric, or electrochemical signal which can be measured by imaging, spectrometric, or mass analysis. In non-limiting examples, the signal generation (8) results from generating lysate of biomolecules for mass spectrometric analysis or genes which can be measured by gene analysis.

In non-limiting embodiments, a specific microwell (1) is selected post analysis of a set of multiple microwells where the analyte (4) is captured by an affinity agent (3) either directly attached to the linkage arm (5) or bound to the linkage arm (5) via a high affinity label and capture agent (11). The linkage arm (5) is further attached to the size exclusion filter (2) in a microwell (1) with an electrode (10). The analyte (4), such as a cell, is captured by an affinity agent (3) either prior to or after the addition of stimuli (7). A signal generating reagent (8) is added to the microwell (1) and the response of the analyte (4) measured. The response is used to select one or more microwells for triggering the release of the analyte (4). A current is applied into the selected microwell (1) with an electrode (10) to break the linkage arm (5) and allow release of the analyte (4) through the size exclusion filter (2).

In non-limiting embodiments or examples, variations in the pore size of the size exclusion filter (2) are of sufficient size to only pass, for example, a lysate, a cell, or multiple cells. In non-limiting examples, the pore is of a circular shape or of a slot shape. The size exclusion filter (2) can be a porous matrix which is solid or semi-solid material, and may be comprised of an organic or inorganic, water insoluble material. In non-limiting examples, cell capture is compatible with growing cells in a microwell (1). In non-limiting examples, the size exclusion filter (2) removes media but retains analyte (4). In non-limiting examples, different stimuli (7) can be added to all microwells (1) of a set of multiple microwells (1). In non-limiting examples, different stimuli (7) can be added to different individual microwells (1) to allow a sub-set of a larger set of multiple microwells (1) with cells to measure additional stimuli response. In non-limiting examples, the microwells (1) can each be loaded with a different cell type. In non-limiting examples, the microwells (1) can be loaded with one cell type, organoid, or a mixture of cell types for co-culturing. Additionally, any one microwell (1) can be loaded with one cell or multiple cells such as those used in co-culturing.

In non-limiting embodiments or examples, cell culture methods commonly used to generate cellular in-vitro responses may be used. The cells are grown in culture plates, wells, or flasks in culture media favorable to cell growth. Growth media selection is common practice to those skilled in the art and is based on both natural and synthetic 7iochemical and can contain, or be free of, serum. Culture media may contain one or more of salts, sugars, amino acids, and vitamins, optionally with a supplement of plasma, which provides growth factors, hormones, albumin, and transferrin. Non-limiting examples of synthetic media such as Eagle's Minimal Essential Medium (MEM) or its modification by Dulbecco (DMEM) are often employed. The cells and growth media are sampled to determine biomolecules and response to biologically active compounds.

In non-limiting embodiments or examples, cell growth and isolation may occur on a size exclusion filter and a microwell (1). Additionally, size exclusion membranes may be used for separation of cells from non-cellular material such as growth media. Grown cells may be isolated in assays in with sensor microwells as shown in Pugia et al. 63/006,833, 63/089,286, and 63/089,308, which are incorporated by reference in their entireties, including the method of FIG. 1.

Non-limiting examples of heterogenic cells include, by way of illustration and not limitation, different cell types, extra-cellular materials, and structures, and originate biological samples or non-biological samples. The heterogenic cells can be natural or synthetic cells. The heterogenic cells can be isolated from organisms such as cells from animals, plants, or be organisms themselves. The heterogenic cells may be malignant or diseased cells such as neoplastic cells, malignant neoplasms, or cancer cells; tumor organoids, circulating endothelial cells; circulating tumor cells; circulating cancer stem cells; cancer mesenchymal cells; and the like. The heterogenic cells may be immune cells such as white blood cells, B cells, T cells, macrophages, NK cells, monocytes, antigen presenting cells (APC), dendritic cells, eosinophils, and granulocytes. The heterogenic cell populations may be from a part of the tissue such as an islet. The heterogenic cell may be of man-made origins, such as antibody-producing cells such as, hybridoma, splenocytes (B-cells), rabbit B-cell fusions, Cho cells, yeast cells, bacteria, and plasma cells. Cells may be of man-made origins, such as induce pluripotent cells or others. The heterogenic cells may be from biological samples of a mammalian subject or a non-mammalian subject, such as tissue or organs. The mammalian subjects may be humans or other animal species.

Biomolecule measurements of cellular response can be indicative of a particular population of cells that can be defined as a cellular phenotype or genotype. Cellular phenotype refers to a group of cells expressing a same group of biomolecules that define a relevant characteristic of the cells such as, but not limited to, an ability to cause disease, an ability to grow, by comprising of specific proteins of the cell, or an ability to produce Biochemical, for example. Cellular genotypes to a group of cells with the same genetic code may be defined by a relevant nucleic acid gene sequence.

The presence, absence, and/or production of biomolecules is used to measure a cellular phenotype where the biomolecule is common to all of the cells of the group and where the biomolecule is specific for the group of cells. This includes biomolecules that can be employed as markers of a phenotype and related to cellular patterns such as, for example, protein expression patterns, nucleic acid mutations, nucleic acid expression patterns, glycan expression patterns, lipid expression patterns, and small molecules secretion, for example.

In non-limiting examples, the biomolecule measurements of cellular response can be used for diagnosis of diseases. In other non-limiting examples, biomolecules can be measured in the presence of one or more compounds to determine a therapeutic response. In other non-limiting examples, measurement of biomolecules produced by cells can be used for determining conditions for increasing or decreasing biomolecule production.

The invention can utilize the many assay methods for biomolecule measurements including the use of attached alkaline phosphatase for the optical immunoassays (OP-IA), electrochemical immunoassay (EC-IA), attached mass labels for mass spectrometric immunoassay (MS-IA), and gene analysis as previously described (Pugia Anal Chem 2021). In a non-limiting example, alkaline phosphatase is used to generate para-amino phenol as the electrochemical reporter from para-amino-phenyl phosphate, mass labels are generated from nanoparticles as the mass spectrometric reporter is measured and genes from cell lysate. Means to use these reagent methods to collect and analyze samples by initially reporting EC-IA results and by later reporting re-testing to provide MS-IA results using a size exclusion filtration membrane to allow cells to be loaded and assayed in with sensor microwells as shown in Pugia et al. 63/006,833, 63/089,286, and 63/089,308; and incorporated herein by reference.

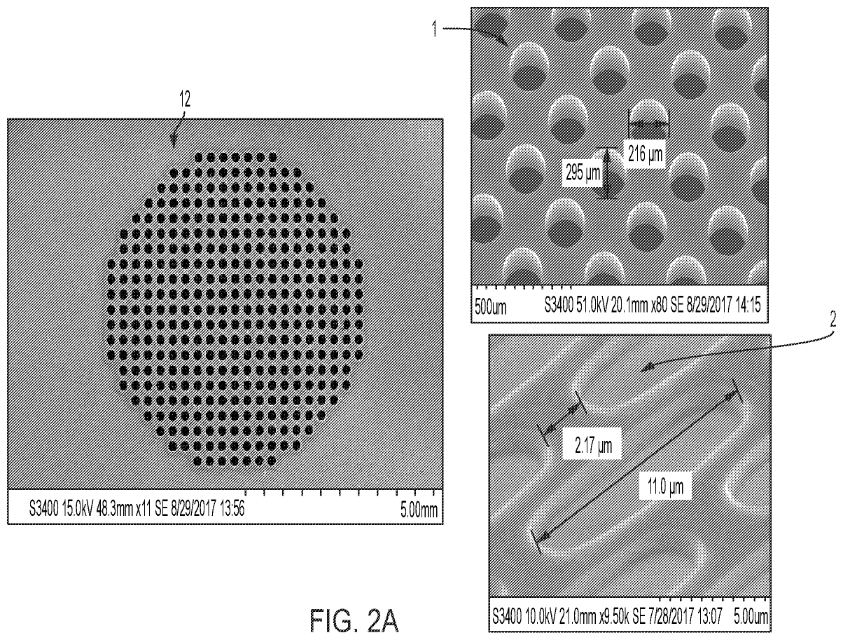

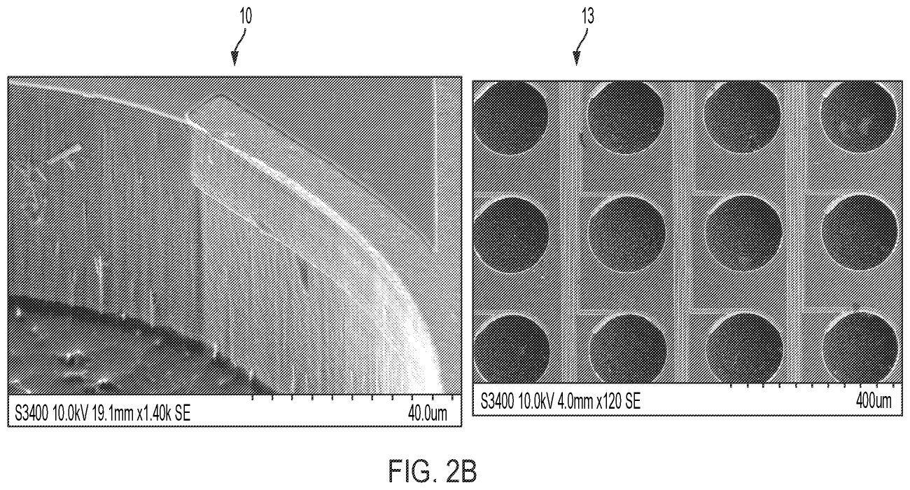

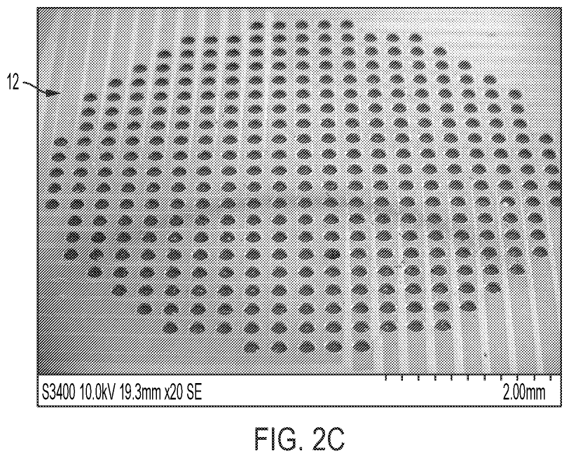

FIGS. 2A-2C show a micro-filtration sensor (12), which is a set of multiple microwells (1), each including a size exclusion filter (2) at the bottom having slotted pores of sufficient pore size to allow released analyte (4) to readily pass under low hydrodynamic force without clogging with debris. Each microwell (1) is connected to an electrode (10) in the microwell (1) via separated current line (13) which connects to leads for changing voltage of any individual microwell (1). The bottom of the slotted size exclusion filter (2) is coated in gold to allow application of reference current and measuring the electrochemical signal in the microwells (1). Assay signals are read by respective current signal vs. voltage in accordance with a non-limiting embodiment of the invention for electrochemical detection or, optically, by imaging. Application of current into the microwell (1) across the bottom slotted size exclusion filter (2) to the electrode (10) in a microwell (1) is used to break the linkage arm (5) for any individual microwell (1).

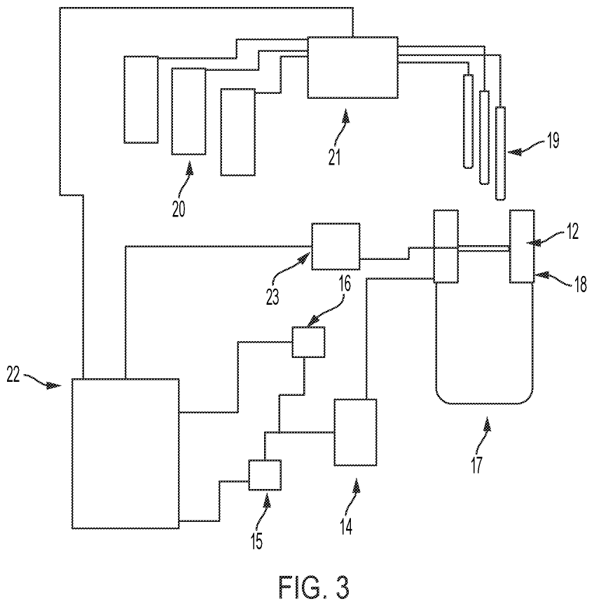

FIG. 3 shows a schematic view of a system using a vacuum pump (14) connected to a vacuum pump motor driver (15) and a pressure sensor (16) as the hydrodynamic force for capturing cells and biomolecules from a sample on a micro-filtration sensor (12) with analyte detection microwells (1) and size exclusion filters (2). The micro-filtration sensor (12) is sealed to a sample collection area (17) using a holder (18) for the micro-filtration sensor (12). The system included fluidics for reactions with liquid reagents with dispensers (19) positioned over the micro-filtration sensor (12) and feeds liquid reagents (20) by programmable dispensing pumps (21). The vacuum pump motor driver (15), pressure sensor (16), and the programmable dispensing pumps (21) are connected to a programmable controller board (22) used to monitor and regulate vacuum pressure for filtration by maintaining a user-defined pressure in the waste collection tube and dispensing liquid reagents by the programmable dispensing pumps (21). Voltage is applied to electrodes in the micro-filtration sensor (12) and the current is read using as electrochemical signals using a potentiostat (23) to read the sensors and to measure voltage and current across working and reference/counter microelectrodes in the microwell (1).

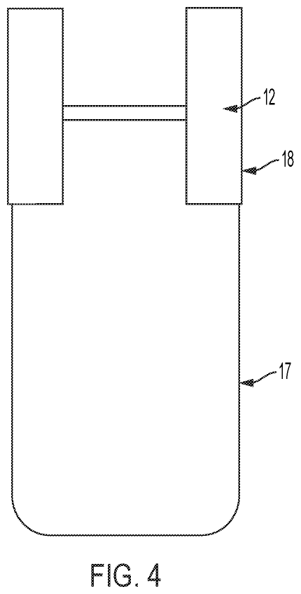

FIG. 4 shows a schematic view of a holder (18) which is capable of holding and reacting fluids over the micro-filtration sensor (12) and also includes a sample collection area (17) that allows collection of the release of the analyte (4) through the micro-filtration sensor (12) when hydrodynamic force is applied.

### Example 1: Method for Isolating and Releasing of Target Analytes from Complex Samples

### Materials:

| | |
|---|---|
| Micro- | Containing 200 individual microwells (1) of 200 μm |
| filtration | diameters and 300 μm depths were made using standard |
| sensor | microfabrication photolithography techniques. |
| Human cells | Breast cancer cells (SKBR3, HTB-30) and hybridoma cells |
| and | producing Her2/nue-specific monoclonal antibodies (mAbs) |
| antibodies | (clone NB3, HB-10205 and clone TA-1, HB-10206) were |
| | acquired from the American Type Culture Collection |
| | (Manassas, VA, USA). |
| Bacterial | Polyclonal antibodies recognizing S. aureus (Thermo Fisher |
| cells and | Scientific), E. coli (MyBioSource, San Diego, CA, USA), |
| antibodies | K. pneumoniae (Thermo Fisher Scientific) and |
| | P. aeruginosa (Abcam, Cambridge, UK). |
| Attachment | Antibodies separately conjugated to alkaline phosphatase |
| of reagents | (ALP) (Thermo Fisher Scientific) using the FastLink |
| to affinity | ALP kit (Abnova, Taipei City, Taiwan), to gold |
| agents | nanoparticle (NP) of 5 to 100 nm diameter (Sigma Aldrich), |
| | and to biotin-PEG4 and to Dylight 488 using the EZ-Link |
| | NHS-conjugation kits (Thermo Fisher Scientific). The |
| | resultant antibody conjugates were stored at 4° C. |
| Signal | The ALP signal generating reagent used an electrochemical |
| generating | solution of 1.05 mM solution of p-aminophenyl phosphate |
| reagent (8) | (pAPP, 3.0 mg, MW 189) in 100 mM TRIS, 600 mM |
| | NaCl, and 5 μM MgCl2 adjusted to pH 9.0. The NP signal |
| | generating reagent used an electrochemical solution of 5 |
| | mM potassium ferricyanide, 5 mM potassium ferrocyanide, |
| | 100 mM Tris-HCl pH 8, and 0.2% Tween-20. |

Unless otherwise noted all other materials were purchased from Sigma Aldrich or Thermo Fisher Scientific.

### Example Method to Demonstrate Making Surface Capture in Microwells:

FIGS. 1 and 2 illustrate a non-limiting embodiment of the present disclosure used for this example, whereby the method utilizes a micro-filtration sensor (12) which is an array of analyte detection microwells (1) each with a size exclusion filter (2) at the bottom (9.0×21.0 μm slot pores) with an overall pore space of 58 mm\hat{ }2 per well (as shown in FIG. 2) and sized to pass cells not selectively bound to the size exclusion filter (2). Additionally, each microwell (1) has an electrode (10) connected by a current line (13) for routing the current into any given microwell (1) (as shown in examples of FIG. 2). This microelectrode filtration membrane in the microwell array was fabricated in silicon dioxide (SiO₂) by double polishing the silicon wafer base substrate (300 μm thick) then introducing the micro-filtration sensor (12) area of 6.5 mm diameter to contain 200 microwells (200 μm diameter, 300 μm deep).

The microfabrication photolithography techniques achieved a<0.1 μm dimensional tolerance. In brief, film layers (4 to 20 μm) of dense, high-quality thermal SiO₂ were patterned with a slotted grid (9.0×21.0 μm pores) by photolithography and dry etch processes to create a size exclusion filter layer. A second layer of 300 μm thickness was made into 200 μm wells with silicon by photolithography and dry etch processes to create the array of microwells. The fabricated microwells layer was then mounted on a size exclusion filter layer. The “bottom side” was further processed for counter electrode lines with copper via electroplating. A 200-nm layer of gold was added to the slotted size exclusion filter by vapor deposition to serve as a gold electrode. Finally, a protective layer of silicon oxide was deposited and patterned to keep contact pads and microwells open.

The gold electrode on the slotted size exclusion filter (2) functionalized with neutravidin through a cleavable S—S bond linkage arm. This linkage arm (5) allows a high affinity capture surface for biotinylated affinity agents. The capture of immunoassay complexes of cell or biomarkers analytes occurs through the linkage arm (5). The functionalization procedure to attach neutravidin through a cleavable S—S bond linkage arm starts by functionalizing with 1 mM of 11-amino-1-undecanethiol hydrochloride in 50 mM phosphate buffer solution (pH=10) to form a self-assembled monolayer by immersion for 17 h with terminal amine groups. Neutravidin was attached using 7.5 mg of N-succinimidyl 3-(2-pyridyldithio)propionate (SPDP) dissolved in 1.6 mL dimethyl sulfoxide, by adding 75 μL of this SPDP solution to 0.1 mL phosphate buffered saline (PBS) applying to the size exclusion filter (2) for 30 minutes at room temperature with vigorous shaking at 75 rpm. The size exclusion filter (2) was next washed with 1.0 mL PBS three times and air dried. Finally, Neutravidin was attached using a solution of 1 mg in 1.0 mL PBS added to the washed SPDP activated size exclusion filter (2). The size exclusion filter (2) was reacted for overnight at room temperature with vigorous shaking at 75 rpm. The size exclusion filter (2) was next washed with 1.0 mL PBS three times and air dried. The linker arm with attached neutravidin (5) contained a disulfide cleavable bond as show in FIG. 1 placed in the 3-(2-pyridyldithio)propionate group.

Functionalized micro-filtration sensors were blocked with 200 μL solution of blocking buffer. The blocking buffer was made with 112.5 mL of water containing 10% Candor (Candor Bioscience, Cat. #110125), 3.18 g MOPSO, 1.50 g BSA (Bovine Serum Albumin (Fraction V), and 60 uL Proclin 200 and pH was adjusted to 7.5 with 10 N sodium hydroxide and the buffer. After blocking overnight, the micro-filtration sensors were washed five times with 200 μL of TBS-T (Tris buffered saline with 0.05% Tween-20) and allowed to air dry.

### Method to Capture Cell and Biomarkers on Surface in Microwells:

FIG. 3 illustrates a non-limiting embodiment of the present disclosure used in the example, whereby the size exclusion filter (2) in sensor microwells (1) is used as part of an analyzer. The analyzer used a vacuum filtration driven by an Arduino based proportional-integral-derivative (PID) controller board (22) with logic to maintain the desired pressure in the sample collection area. It also drives the sample and liquid reagent fluids through a silicon dioxide (SiO₂) microelectrode filtration membrane in microwells (1), in a holder (18). The analyzer used a vacuum as the hydrodynamic force for capturing cells and/or biomolecules on to the functionalized size exclusion filters (2) in the microwells (1) of the micro-filtration sensor (12). Negative pressure for filtration was provided by a vacuum via the underside of the size exclusion filter (2).

The analyzer included fluidics (19-21) for reactions with reagents and electrodes and electronics (23) for detection of electrochemical signals. An Arduino controller with a menu-driven program (Adafruit Industries, New York, NY, USA) was used as the PID controller board (22) with a motor driver circuit board (15) used to monitor and regulate vacuum pressure for filtration (10-100 mbar negative pressure ±10%). An MPXV5050DP analog differential pressure sensor (16) (Mouser Electronics, Mansfield, TX, USA) was used to measure the pressure in a conical 5-ml Eppendorf tube serving as the sample collection area (17). Arduino-based vacuum-driven fluidic controller board (22) included a proportional-integral-derivative (PID) control to maintain a user-defined pressure in the collection tube. The pump and the pressure sensor were connected to the conical tube using appropriate fluidic connectors (IDEX Health & Science, Oak Harbor, WA, USA). The control loop drives a DC diaphragm vacuum pump (14) (22000.011, Boxer Pumps, Ottobeuren, Germany) through a DRV8838 brushed DC motor driver (Texas Instruments, Dallas, TX, USA) to evacuate air from the 5-ml Eppendorf tube. The liquid dispensing was controlled using the same Arduino controller and three peristaltic pumps (21) with linear actuator motors to pump liquids (20) into a sensor for reagent and sample delivery (100 uL±1%) through needles used as liquid dispensers (19).

The neutravidin linked to the gold surface of the slotted membrane was used to demonstrate cell and biomarker capture using the analyser for the following procedure. Biotinylated antibody reagents for affinity \hat{ }capture (3) and alkaline phosphate (ALP) labelled antibodies for signal generation (8) in buffer are manually added to a complex sample containing the analyte (4) and incubated. The analyzer, as discussed above and shown FIG. 3, was controlled using the logic controller (PID), which was used to automate: 1) drawing complex samples with antibodies and bound analyte (4) into the microwells (1) of the micro-filtration sensor ((12) of FIG. 2A) through turning the vacuum on and off; 2) after incubation of antigen affinity complex in sensor microwells (1) for <5 minutes, the antigen affinity complex was captured by the neutravidin attached through a cleavable S—S bond linkage arm ((5) of FIG. 1) to the size exclusion filter ((2) of FIG. 1) in the microwells (1) followed by addition and removal of wash solutions five times with 200 μL of TBS-T (Tris buffered saline with 0.05% Tween-20) using the peristaltic pump with linear actuator motors to dispense TBS-T and vacuum to remove from the membrane; 4) signal generating reagent (8) with electrochemical solution to generate a signal which is read by the current change in the microwell (1) using the potentiostat circuit board (23) for measurement of current for each microwell (1).

In practice, several different signal generating reagent (8) can be used as the signal generating reagents (10). For example, use of the electrochemical immunoassay (EC-IA), as previously described as an example (Pugia, Anal. Chem. 2021), where enzyme, like alkaline phosphatase, is used to generate redox probe, like para-amino phenol as the electrochemical response from an enzyme substrate, like para-amino-phenyl phosphate, or a nanoparticle is used as electrochemical response from ferrous cyanide or other redox probes as the signal generating reagent (Pugia, papers 1-3). The signal can detected by many electrochemical methods such as impedance spectroscopy (EIS) or square wave voltamerty (SWV) as described in Pugia (See Papers 1-3 Anal Chem 2021).

In this example with ALP as the signal generator, the potentiostat used to read the SWV signal in the microwell (1) also allowed measuring and controlling of the voltage and the current. The potentiostat circuit board (23) allowed measurement of 100 μA to 100 nA current across working and reference/counter microelectrodes in each separate microwell (1) and was controlled through a multiplex board (MUX board). The Arduino controller delivered voltage and current results to a data file for analysis. The device is connected to a computer via a smart card reader from the Arduino controller to provide all data for electrochemical analysis. The necessary hardware and electronics are fitted within a 12×12×6 inches case, including room for waste or sample containment, three types of liquid reagents, the photostat to read the sensors, and a small liquid crystal display (LCD) for the Arduino (PID).

### Example Demonstration of Capture and Detection Cells and Biomolecules in Microwells:

Immunoassay methods for biomolecule measurements with attached alkaline phosphatase and biotin that can be used for the electrochemical immunoassay (EC-IA), as previously described (Pugia Anal Chem 2021), were used. The alkaline phosphatase is used to generate para-amino phenol as the electrochemical signal (9) from para-amino-phenyl phosphate.

Additional reagent methods to analyze samples, such as labels, are generated from nanoparticles, mass spectrometric reporter labels, or optically labeled monoclonal antibodies for signal generation can be used, as previously demonstrated (Pugia Anal Chem 2017, 2019, and 2021). All of these reagents allow using a size exclusion filter (2), and cells to be loaded and assayed in sensor microwells (1) as shown in Pugia et al. 63/006,833, 63/089,286, and 63/089,308, which are incorporated herein by reference for mass spectrometric and gene analysis.

For demonstration of capture of human breast cancer cells, SKBR3 cells were grown in culture and commercial antibodies for said cells were used. Stable samples of SBKR3 cells were prepared as previously described by cold fixation in 2% formaldehyde at 4° C. overnight, followed by washing and storage in the presence of fetal bovine serum. Before use, stock solutions of fixed SKBR3 cells were passed through a 20-μm pluriStrainer mesh filter (pluriSelect, Leipzig, Germany) and counted with a hemocytometer yielding a final cell density of 2×10 4 cells/L. To make the antibody complex for capture, a sample containing 10 μL of SKBR3 cells (1 to 20 cell/μL), 470 μL TCPBS, 10 μL biotin-labeled HER2/neu-specific monoclonal antibody (mAbs) (2.1 mg/mL) (clone NB-3) and 10 μL ALP-labeled HER2/neu-specific monoclonal antibodies (mAbs) (1.6 mg/mL) (clone TA-1) were incubated for 1 hour at room temperature.

For demonstration of capture of bacteria, S. aureus, E. coli or P. aeruginosa were grown in culture and commercial antibodies for said cells were used. Cell lysates were prepared by addition of BPEP-II surfactant. To make the antibody complex for capture, a sample containing 100 μL of the lysate sample (0, 5, 10, 20, 30, and 40 thousand cells or lysate equivalent per assay) was added to 48 μL of the biotinylated S. aureus, E. coli, or P. aeruginosa polyclonal antibodies (0.75 μg/assay) and 30 μL of the same polyclonal antibodies conjugated to ALP (1.50 μg/assay) was incubated for 1 hour at room temperature.

The human cells or bacterial cell lysates with the antibody were introduced directly into the micro-filtration sensor (12) in its holder (18) with its size exclusion filter (2) functionalized with neutravidin and cleavable S—S bond linkage arm (5) produced as described above. The same analyzer as described above was used for; 1) drawing the antibody:antigen complex into the microwells (1) through turning the vacuum on and off; 2) incubating antibody:antigen complex in sensor microwells (1) for <5 for the neutravidin capture in the microwells (1); 3) washing the antibody:antigen complex by addition and removal of wash solutions five times with 200 μL of TBS-T and followed by a vacuum; and 4) detecting the antibody:antigen complex by addition of 150-μL of electrochemical solution containing 1.05 mM solution of p-aminophenyl phosphate (pAPP, 3.0 mg) in 100 mM TRIS, 600 mM NaCl, and 5 μM MgCl₂ adjusted to pH 9.0. Upon addition of the pAPP, electrochemical solution and the electrochemical current (uA) readings from 0 to 0.2 V were obtained for all the wells at 5 minutes of reaction with ALP using the potentiostat circuit board (23) for measurement of 1 μA to 10 nA current for each microwell sensor. Wells with one or more cancer cells were identified producing current change (of >0.1 uA) where wells with no cells did not produce current. Wells with 1000 or more bacterial cells (lysate equivalent) were also identified producing current change (of >0.1 uA) where wells with no cells did not produce current. Confirmation of the presence of cancer cells or bacterial lysate in each microwell (1) was performed by carefully removing the microwell sensor, drying the bottom with Kimwipe, and placing on a glass slide for fluorescent imaging to confirm the lack or presence of the Dylight 488 as a fluorescent signal of the signal antibody.

### Example Rapid Sample Processing Procedure for Release and Isolation of Cells:

Method for release of target analytes (4) from complex samples occurred by reducing the S—S bond in the linkage arm (5) by application of a reducing potential of −0.1 to −10 V in the microwell (1). To further illustrate the embodiment of the present disclosure, one particular well of interest was identified as containing a human cell, and the additional reducing voltage was applied specifically into that well to cause complete S—S cleavage and release of the immune complex. Again, confirmation of the presence of cancer cells or in each microwell (1) was performed by carefully removing the microwell sensor, drying the bottom with Kimwipe, and placing on a glass slide for fluorescent imaging to confirm the lack or presence of the Dylight 488 as a fluorescent signal of the signal antibody. This cleavage did not cause lysis of the cancer cell or damage release of the biomolecules. Whereas microwells lacking the reducing potential did not release biomolecules lysates or cells, thereby demonstrating selective release.

The released biomolecules were spun out of microwells (1) using a microcentrifuge of the sample into a new tube serving as the sample collection area (17). FIG. 4 illustrates an embodiment used to collect the released biomolecules, whereby a holder (18) is capable of holding and reacting fluids over the micro-filtration sensor (12) and also includes a sample collection area (17) that allows collection of release of the analyte (4) from the microwells (1) when hydrodynamic force is applied. This format allowed the micro-filtration sensor to be washed with 200 uL of water and the cells and/or biomolecule released from the microwells (1) surface to end up in the tube for collection.

To demonstrate, selective release of cancer cells or bacterial lysates from a microwell (1) was determined by presence of the released cell or lysate in the sample collection area (17) by optical immunoassays previously disclosed (Pugia Anal Chem 2019 and 2021) and the absence of cell or lysate was determined in the microwell (1) by microscope imaging as described above. This reduction method was able to completely and selectively remove cancer cells or bacterial lysates from selected microwells.

While the invention has been illustrated and described in detail in the drawings and foregoing description, the same is to be considered as illustrative and not restrictive in character, it being understood that only the representative embodiments have been shown and described and that all changes, equivalents, and modifications that come within the spirit of the inventions defined by the claims are desired to be protected. All publications, patents, and patent applications cited in this specification are herein incorporated by reference as if each individual publication, patent, or patent application were specifically and individually indicated to be incorporated by reference and set forth in its entirety herein.

1. A method of determining cellular response comprising:
introducing a complex sample into a microwell, the microwell having a size exclusion filter, the complex sample having an analyte;
introducing a stimuli into the microwell;
measuring a response from the analyte;
analyzing the response; and
releasing the analyte from the microwell.

2. The method of claim 1, further comprising applying a voltage in the microwell, wherein the voltage allows the analyte to exit the microwell via the size exclusion filter, and wherein the analyte comprises cells or biomolecules.

3. The method of claim 2, wherein the response from the analyte triggers applying the voltage in the microwell.

4. The method of claim 1, wherein the analyte is captured by an affinity agent, wherein the affinity agent is connected to a cleavable linkage arm, the cleavable linkage arm connected to the microwell.

5. The method of claim 4, wherein a voltage separates the cleavable linkage arm and the capture agent.

6. The method of claim 1, wherein the analyte is selectively captured in the microwells by an affinity agent prior to exiting the microwell.

7. The method of claim 1, wherein the response from the analyte is measured before adding the stimuli into the microwell.

8. The method of claim 1, further comprising measuring molecules and/or biomolecules from the analyte in the presence of one or more compounds to determine therapeutic response or the biological activity of the analyte.

9. The method of claim 1, further determining conditions for of the analyte that increases or decreases biomolecule production depending on the response of the analyte.

10. The method of claim 1, further comprising diagnosing the analyte depending on the response of the analyte.

11. The method of claim 1, wherein the response comprises a signal from an antibody, biomolecule, or a cell.

12. The method of claim 1, where in a response is measured in the presence of one or more compounds, and wherein the response determines a therapeutic response or the biological activity of the analyte.

13. The method of claim 1, wherein the released analyte is intact.
